(19) United States
(12) Patent Application Publication
(10) Pub. No.: US 2024/0390277 A1
(43) Pub. Date: Nov. 28, 2024

(54) LIQUID FORMULATIONS OF TRAZODONE

(71) Applicant: Rubicon Research Private Limited, Thane West (IN)

(72) Inventors: Pratibha Sudhir PILGAONKAR, Mumbai (IN); Madhav KAMAT, Monmouth Junction, NJ (US); Ashwini Hemant GADKARI, Thane (W) (IN)

(21) Appl. No.: 18/636,185

(22) Filed: Apr. 15, 2024

(30) Foreign Application Priority Data
Apr. 15, 2023 (IN) 202221076972

(51) Int. Cl. A61K 9/08; A61K 9/00; A61K 31/496; A61K 47/02; A61K 47/10; A61K 47/14; A61K 47/18

(52) U.S. Cl. CPC A61K 9/08; A61K 9/0053; A61K 31/496; A61K 47/02; A61K 47/10; A61K 47/14; A61K 47/183

(57) ABSTRACT

The present disclosure relates to stable oral liquid pharmaceutical formulations comprising trazodone or its pharmaceutically acceptable salts thereof, one or more sweeteners, one or more preservatives and water. The formulations of the present disclosure are palatable, alcohol-free, freeze-thaw stable, and buffer-free. The present disclosure further relates to method of treatment of major depressive disorder comprising administering to the subject in need thereof stable oral liquid pharmaceutical formulation comprising trazodone or its pharmaceutically acceptable salts thereof, one or more sweeteners, one or more preservatives and water.

### FIELD OF THE INVENTION

The present disclosure relates to stable oral liquid pharmaceutical formulations comprising trazodone or pharmaceutically acceptable salts thereof, one or more sweeteners, one or more preservatives and water. The formulations of the present disclosure are palatable, alcohol-free, freeze-thaw stable, and buffer-free. The present disclosure further relates to method of treatment of major depressive disorder comprising administering to the subject in need thereof stable oral liquid pharmaceutical formulation comprising trazodone or pharmaceutically acceptable salts thereof, one or more sweeteners, one or more preservatives and water.

### BACKGROUND

Trazodone hydrochloride is indicated for the treatment of depression. The efficacy of trazodone has been demonstrated in both inpatient and out-patient settings and for depressed patients with and without prominent anxiety. Major Depressive Episode is a form of depression and implies a prominent and relatively persistent (nearly every day for at least 2 weeks) depressed or dysphoric mood that usually interferes with daily functioning, and includes at least four of the following eight symptoms: change in appetite, change in sleep, psychomotor agitation or retardation, loss of interest in usual activities or decrease in sexual drive, increased fatigability, feelings of guilt or worthlessness, slowed thinking or impaired concentration, and suicidal ideation or attempts.

Trazodone was developed in the 1960s as a second-generation antidepressant. In contrast to most other antidepressants available at the time of its development, trazodone showed minimal effects on muscarinic cholinergic receptors. Trazodone was approved by the United States Food and Drug Administration in 1981 and was the first non-tricyclic antidepressant approved in the United States.

The mechanism of action of trazodone is not fully understood. The structure of trazodone includes a triazolo moiety that may impart antidepressant effects. Trazodone's primary metabolite, m-chlorphenylpiperazine (m-cpp), is a potent 5-hydroxytryptophan (5-HT) antagonist. In animals, trazodone is believed to selectively inhibit serotonin uptake by brain synaptosomes and potentiates the behavioral changes induced by the serotonin precursor, 5-hydroxytryptophan (5-HT). Cardiac conduction effects of trazodone in the anesthetized dog are qualitatively dissimilar and quantitatively less pronounced than those seen with tricyclic antidepressants. Trazodone is not a monoamine oxidase inhibitor and, unlike amphetamine-type drugs, does not stimulate the central nervous system. In humans, trazodone hydrochloride is well absorbed after oral administration without selective localization in any tissue. Trazodone is highly protein bound (89% to 95%) and has a moderate volume of distribution (1.2 L/kg). Trazodone is rapidly and extensively metabolized in the liver by hydroxylation, dealkylation, and N-oxidation.

The oral tablets dosage of Trazodone is initiated at a low level and increased gradually, noting the clinical responses and any evidence of intolerance. Once an adequate response has been achieved, trazodone total dosage amounts are generally gradually reduced, with subsequent adjustment depending on the therapeutic response.

### SUMMARY

The present disclosure relates to stable oral liquid pharmaceutical formulations comprising trazodone or pharmaceutically acceptable salts thereof, one or more sweeteners, one or more preservatives and water. The formulations of the present disclosure are palatable, alcohol-free, freeze-thaw stable, and buffer-free. The present disclosure further relates to method of treatment of major depressive disorder comprising administering to the subject in need thereof stable oral liquid pharmaceutical formulation comprising trazodone or pharmaceutically acceptable salts thereof, one or more sweeteners, one or more preservatives and water.

In certain embodiments, the liquid pharmaceutical formulation comprises: (i) trazodone or a pharmaceutically acceptable salt thereof; (ii) a non-polymeric cosolvent; (iii) a pH adjuster; and (iv) water; wherein the formulation does not comprise polyethylene glycol, and wherein the pH of the formulation is about 2.5 to about 5.5.

In certain embodiments, the trazodone is trazodone hydrochloride. In certain embodiments, the trazodone is about 0.1% w/v to about 10% w/v of the formulation. In certain embodiments, the trazodone is about 0.5% w/v to about 5% w/v of the formulation. In certain embodiments, the trazodone is about 0.8% w/v to about 1.2% w/v of the formulation.

In certain embodiments, the non-polymeric cosolvent is polyol. In certain embodiments, the non-polymeric cosolvent comprises propylene glycol, glycerine, or combinations thereof.

In certain embodiments, the non-polymeric cosolvent is about 1% w/v to about 50% w/v of the formulation. In certain embodiments, the cosolvent is about 3% w/v to about 30% w/v of the formulation.

In certain embodiments, the formulation does not comprise a polymeric cosolvent.

In certain embodiments, the non-polymeric cosolvent comprises propylene glycol. In certain embodiments, the propylene glycol is about 1% w/v to about 15% w/v of the formulation.

In certain embodiments, the non-polymeric cosolvent comprises glycerine. In certain embodiments, the glycerine is about 2% w/v to about 30% w/v of the formulation.

In certain embodiments, the non-polymeric cosolvent comprises a combination of propylene glycol and glycerine.

In certain embodiments, the pH adjuster is an acid. In certain embodiments, the acid has at least one pKa value of less than 5. In certain embodiments, the acid has at least one pKa value of about 1.0 to about 3.7.

In certain embodiments, the pH adjuster is a phosphoric acid. In certain embodiments, the phosphoric acid is ortho-phosphoric acid.

In certain embodiments, the pH adjuster is about 0.01% w/v to about 3% w/v of the formulation. In certain embodiments, the pH adjuster is about 0.01% w/v to about 0.5% w/v of the formulation. In certain embodiments, the pH of the formulation is about 3.8 to about 4.8.

In certain embodiments, the formulation further comprises at least one pharmaceutically acceptable excipient. In certain embodiments, at least one pharmaceutically acceptable excipient comprises a preservative, an antioxidant, a chelating agent, a sweetener, a flavoring agent, a coloring agent, a thickening agent, or combinations thereof.

In certain embodiments, the antioxidant comprises butylatedhydroxy anisole (BHA), butylatedhydroxy toluene (BHT), sodium metabisulfite, ascorbic acid, propyl gallate, or combination thereof. In certain embodiments, the antioxidant comprises propyl gallate.

In certain embodiments, the chelating agent comprises disodium edetate, dipotassium edetate, edetic acid, sodium edetate, trisodium edetate, or a combination thereof. In certain embodiments, the chelating agent comprises disodium edetate.

In certain embodiments, the preservative comprises sodium benzoate, methyl paraben, propylparaben, or combinations thereof.

In certain embodiments, the sweetener is sorbitol, sucralose, acesulfame potassium, sodium saccharin, or combinations thereof.

In certain embodiments, the formulation is stable for greater than 18 months.

In certain embodiments, the formulation is freeze-thaw stable.

In certain embodiments, the liquid pharmaceutical formulation comprises: (i) about 0.5% to about 2% trazodone hydrochloride; (ii) about 5% to about 15% glycerine; (iii) about 3% to about 15% propylene glycol; (iii) a phosphoric acid; and (iv) water; wherein the formulation does not comprise a polymeric cosolvent and wherein the pH of the formulation is about 3.8 to about 4.8.

In certain embodiments, the liquid pharmaceutical formulation comprises: (i) about 1% trazodone hydrochloride; (ii) about 15% glycerine; (iii) about 7% propylene glycol; (iv) a phosphoric acid; and (v) water; wherein the formulation does not comprise a polymeric cosolvent and wherein the pH of the formulation is about 3.8 to about 4.8.

In certain embodiments, the liquid pharmaceutical formulation comprises: (i) about 1% trazodone hydrochloride; (ii) about 10% glycerine; (iii) about 5% propylene glycol; (iv) a phosphoric acid; and (v) water; wherein the formulation does not comprise a polymeric cosolvent and wherein the pH of the formulation is about 3.8 to about 4.8.

In certain embodiments, the formulation comprises 100 mg/ml trazodone, and wherein the formulation is stable for at least 12 months. In certain embodiments, the formulation comprises 100 mg/ml trazodone, and wherein the formulation is stable for at least 18 months. In certain embodiments, the formulation comprises 100 mg/ml trazodone, and wherein the formulation is stable for at least 24 months.

In certain embodiments, the formulation comprises 100 mg/ml trazodone, and wherein the formulation provides a Cₘₐₓ of about 500 ng/mL to about 5000 ng/ml when administered to a subject under fed condition.

In certain embodiments, the formulation comprises 100 mg/ml trazodone, and wherein the formulation provides a AUC₀₋ₜ of about 8000 ng*hr/mL to about 54000 ng*hr/mL when administered to a subject under fed condition. In certain embodiments, the formulation comprises 100 mg/ml trazodone, and wherein the formulation provides a AUC_{0-∞}, of about 9000 ng*hr/mL to about 100000 ng*hr/mL when administered to a subject under fed condition. In certain embodiments, the formulation comprises 100 mg/ml trazodone, and wherein the formulation provides Tₘₐₓ of about 0.1 hours to about 8 hours when administered to a subject under fed or fasting conditions.

In certain embodiments, the present disclosure relates to a method of treating a depressive disorder in a subject in need thereof, the method comprising administering a liquid pharmaceutical formulation comprising: (i) trazodone or a pharmaceutically acceptable salt thereof; (ii) a cosolvent; (iii) a pH adjuster; and (iv) water; wherein the formulation does not comprise polyethylene glycol, and wherein the pH of the formulation is about 2.5 to about 5.5. In certain embodiments, the depressive disorder is a major depressive disorder.

In certain embodiments, the liquid pharmaceutical formulation is administered at a dose of about 25 mg/day to about 700 mg/day trazodone. In certain embodiments, the liquid pharmaceutical formulation is administered at a dose of about 50 mg/day to about 650 mg/day trazodone. In certain embodiments, the liquid pharmaceutical formulation is administered at a dose of about 75 mg/day to about 650 mg/day trazodone. In certain embodiments, the liquid pharmaceutical formulation is administered at a dose of about 100 mg/day to about 600 mg/day trazodone.

In certain embodiments, the present disclosure relates to a method of treating a disease or condition responsive to 5-hydroxy tryptamine (5-HT) receptor antagonist in a subject in need thereof, the method comprising administering a liquid pharmaceutical formulation comprising: (i) trazodone or a pharmaceutically acceptable salt thereof; (ii) a cosolvent; (iii) a pH adjuster; and (iv) water; wherein the formulation does not comprise polyethylene glycol, and wherein the pH of the formulation is about 2.5 to about 5.5.

In certain embodiments, the liquid pharmaceutical formulation is administered at a dose of about 5 mg/day to about 650 mg/day trazodone. In certain embodiments, the liquid pharmaceutical formulation is administered at a dose of about 10 mg/day to about 600 mg/day trazodone.

In certain embodiments, the disease or condition responsive to 5-hydroxy tryptamine (5-HT) receptor antagonist comprises depression, anxiety, Alzheimer disease, substance abuse, bulimia, fibromyalgia, post-traumatic stress disorder (PTSD), sleep disorders, insomnia, benzodiazepine and/or alcohol dependence or abuse, dementia, schizophrenia, chronic pain, diabetic neuropathy, attention deficit hyperactivity disorder, autism spectrum disorder, or obsessive-compulsive disorder.

In certain embodiments, the liquid pharmaceutical formulation is administered once or more than once daily. In certain embodiments, the liquid pharmaceutical formulation is administered in fed state. In certain embodiments, the liquid pharmaceutical formulation is administered in within 2 hours of bedtime. In certain embodiments, the liquid pharmaceutical formulation is administered through the day in divided doses.

In certain embodiments, about 10% to about 75% of the total daily liquid pharmaceutical formulation is administered in the morning, and about 25% to about 90% of the total daily liquid pharmaceutical formulation is administered to the subject in the evening. In certain embodiments, about 10% to about 50% of the total daily liquid pharmaceutical formulation is administered in the morning, and about 40% to about 90% of the total daily liquid pharmaceutical formulation is administered to the subject in the evening.

In certain embodiments, the liquid pharmaceutical formulation is administered for a period of time, wherein the daily amount of trazodone administered to the subject is adjusted during the period of time. In certain embodiments, the daily amount of trazodone is increased every 2-5 days as needed during the period of time. In certain embodiments, the daily amount of trazodone is increased every 3-4 days as needed during the period of time. In certain embodiments, the daily amount of trazodone is decreased every 2-5 days as needed during the period of time. In certain embodiments, the daily amount of trazodone is decreased every 3-4 days as needed during the period of time. In certain embodiments, the daily amount of trazodone is increased every 3-4 days as needed during the period of time, and then decreased every 3-4 days as needed during the period of time.

In certain embodiments, the subject is administered a liquid pharmaceutical formulation comprising not more than 150 mg/day trazodone for first period of time, and then administered a liquid pharmaceutical formulation comprising not less than 150 mg/day trazodone for a second period of time. In certain embodiments, the daily amount of trazodone administered to the subject increases 5 mg/day to 100 mg/day every 1-5 days as needed during the second period of time until a maximum effective dose is achieved. In certain embodiments, the daily amount of trazodone administered to the subject increases about 10 mg/day to 75 mg/day every 1-5 days as needed during the second period of time until a maximum effective dose is achieved. In certain embodiments, the daily amount of trazodone administered to the subject increases about 10 mg/day to 50 mg/day every 1-5 days as needed during the second period of time until a maximum effective dose is achieved.

In certain embodiments, the subject is administered a liquid pharmaceutical formulation comprising trazodone for a third period of time after the second period of time. In certain embodiments, the daily amount of trazodone administered to the subject decreased about 5 mg/day to 100 mg/day every 1-5 days as needed during the third period of time until a minimum effective dose is achieved. In certain embodiments, the daily amount of trazodone administered to the subject decreased about 10 mg/day to 75 mg/day every 1-5 days as needed during the third period of time until a minimum effective dose is achieved.

In certain embodiments, the minimum effective dose is less than 150 mg/day trazodone. In certain embodiments, the minimum effective dose is less than 50 mg/day trazodone.

### DETAILED DESCRIPTION

The present disclosure provides stable oral liquid pharmaceutical formulations comprising trazodone or pharmaceutically acceptable salts thereof, one or more sweeteners, one or more preservatives and water. The present disclosure further relates to method of treatment of major depressive disorder comprising administering to the subject in need thereof stable oral liquid pharmaceutical formulation comprising trazodone or pharmaceutically acceptable salts thereof, one or more sweeteners, one or more preservatives and water. In one embodiment, the formulations of the present disclosure are palatable, alcohol-free, freeze-thaw stable, and buffer-free. In another embodiment the formulations of the present disclosure have a shelf-life of at least 12 months, 18 months, 24 months, 30 months, or 36 months.

Unless defined otherwise, all technical and scientific terms used herein have the same meanings as commonly understood by one of ordinary skill in the art.

In some embodiments, the dosage form of the present disclosure is a stable, oral liquid dosage form. A liquid dosage can be advantageous as it can be suitably adjusted to meet the dosing needs of the patients. Such a liquid dosage form can provide significant benefits over the solid tablet dosage forms where titration is required, e.g., when trazodone is initiated at a low level or dose and increased gradually, or alternatively, after an adequate response has been achieved and the trazodone total dosage amounts are gradually reduced. Liquid dosage forms allow for more precise and accurate dose adjustment. Liquid dosage forms can be more convenient, allowing administration of the exact amount of trazodone desired and/or required. Liquid dosage forms are particularly useful with elderly depressed patients, or patients suffering from dysphagia. Liquid dosage forms can also be useful in the case of severely ill or incapacitated patients needing medications administered through gastric or nasal tubes.

No trazodone liquid formulations, e.g., oral solution, have been developed in the US market. Some of the possible reasons for difficulty in developing an oral solution of trazodone are:
1) instability of trazodone in aqueous solutions resulting in oxidative degradation impurities, undesirable discoloration of the solutions over time and a consequent reduced shelf-life of a liquid product;
2) incompatibility with ethanol; and
3) pH dependent solubility of trazodone that can result in precipitation of the active due to any fluctuations in the pH of the liquid product.

Additionally, liquid formulations also need to be highly palatable to be acceptable and patient compliant. The present disclosure provides for liquid formulations of trazodone which overcome one or more of these difficulties.

Previously, liquid dosage forms of trazodone were reported. U.S. Pat. No. 10,292,931 discusses a concentrated trazodone drops formulation that has a pH value between 5.0 and 6.0 and comprises at least two cosolvents selected from the group comprising glycols and polyglycols.

In some embodiments, the present disclosure provides a liquid dosage form for oral administration that is aimed to fulfil this unmet medical need of liquid trazodone. In some embodiments, the present disclosure successfully overcomes the limitations of prior disclosures and unmet medical needs, and provides a chemically and physically stable, pleasingly palatable, and manufacturable solution product for oral use in patients. The present disclosure offers technical advancement and has economic significance for elderly, and the needful patient community. The formulations of the present disclosure are palatable, alcohol-free, freeze-thaw stable, and buffer-free and have a shelf-life of at least 12 months 18 months, 24 months, 30 months, or 36 months.

In one embodiment, the formulations of the present disclosure comprise the active agent trazodone or pharmaceutically acceptable salts thereof. In a further embodiment, the formulations of the present disclosure comprise trazodone hydrochloride. In another embodiment, the formulation of the present disclosure comprises any known polymorphic form of trazodone or pharmaceutically acceptable salts thereof.

In one embodiment, the formulation of the present disclosure comprises about 0.1% w/v to about 10% w/v trazodone. In some embodiments, the formulation of the present disclosure comprises about 0.2% w/v to 8% w/v trazodone. In some embodiments, the formulation of the present disclosure comprises about 0.5% w/v to about 5% w/v trazodone. In some embodiments, the formulation of the present disclosure comprises about 0.8% w/v to about 1.2% w/v trazodone. In some embodiments, the formulations of the present disclosure comprise about 0.5% w/v to about 4% w/v, about 0.5% w/v to about 3% w/v, about 0.5% w/v to about 2% w/v, or about 0.5% w/v to about 1.5% w/v trazodone. In some embodiments, the formulations of the present disclosure comprise about 0.5% w/v, about 0.75% w/v, about 1% w/v about 1.25% w/v, or about 1.5% w/v trazodone. In some embodiments, the formulations of the present disclosure comprise about 1% w/v trazodone. In one embodiment, the formulations of the present disclosure comprise therapeutically effective amount of trazodone.

In some embodiments, the formulation of the present disclosure comprises about 10 mg/ml to about 200 mg/ml trazodone. In some embodiments, the formulation of the present disclosure comprises about 50 mg/ml to about 150 mg/ml trazodone. In some embodiments, the formulation of the present disclosure comprises about 75 mg/ml to about 125 mg/ml trazodone. In some embodiments, the formulation of the present disclosure comprises about 10 mg/ml, about 25 mg/ml, about 50 mg/ml, about 75 mg/ml, about 100 mg/ml, about 125 mg/ml, about 150 mg/ml, about 175 mg/ml, or about 200 mg/ml trazodone.

In some embodiments, the formulation of the present disclosure comprises water. In some embodiments, water is a liquid carrier or solvent of the formulation. In some embodiments, the formulation comprises water in quantity sufficient to make up the volume to 100%.

In another embodiment, the formulation of the present disclosure comprises at least one cosolvent. In some embodiments, the formulation comprises a carbon-based cosolvent, i.e., a cosolvent comprising one or more carbon atoms. In some embodiments, the formulation comprises a non-polymeric carbon-based cosolvent. In some embodiments, the non-polymeric cosolvent is a polyol. In some embodiments, the formulation of the present disclosure comprises at least one cosolvent selected from, but not limited to, propylene glycol, glycerine, or combinations thereof. In another embodiment, the formulation of the present disclosure does not comprise a polyglycol. In a further embodiment, the formulation of the present disclosure does not comprise a polyethylene glycol. The present disclosure demonstrates that oral liquid formulations with the described cosolvents, but without polymeric, carbon-based cosolvents, provide for increased chemical and/or freeze-thaw stability of trazodone oral solution formulations.

In one embodiment, the formulation of the present disclosure comprises about 1% w/v to about 50% w/v cosolvent. In some embodiments, the formulation of the present disclosure comprises about 2% w/v to about 40% w/v cosolvent. In some embodiments, the formulation of the present disclosure comprises about 3% w/v to about 30% w/v cosolvent. In some embodiments, the formulation of the present disclosure comprises about 3% w/v to about 25% w/v cosolvent.

In some embodiments, the formulation of the present disclosure comprises two cosolvents, wherein about 1% w/v to about 50% w/v of the formulation is cosolvent. In some embodiments, the formulation of the present disclosure comprises two cosolvents, wherein about 2% w/v to about 40% w/v of the formulation is cosolvent. In some embodiments, the formulation of the present disclosure comprises two cosolvents, wherein about 3% w/v to about 30% w/v of the formulation is cosolvent. In some embodiments, the formulation of the present disclosure comprises two cosolvents, wherein about 3% w/v to about 25% w/v of the formulation is cosolvent. In some embodiments, the formulation does not comprise a polymeric, carbon-based solvent, e.g., a polyglycol.

In some embodiments, the formulation of the present disclosure comprises two or more cosolvents, wherein about 1% w/v to about 50% w/v of the formulation is cosolvent. In some embodiments, the formulation of the present disclosure comprises two or more cosolvents, wherein about 2% w/v to about 40% w/v of the formulation is cosolvent. In some embodiments, the formulation of the present disclosure comprises two or more cosolvents, wherein about 3% w/v to about 30% w/v of the formulation is cosolvent. In some embodiments, the formulation of the present disclosure comprises two or more cosolvents, wherein about 3% w/v to about 25% w/v of the formulation is cosolvent. In some embodiments, the formulation does not comprise a polymeric, carbon-based solvent, e.g., a polyglycol.

In some embodiments, the cosolvent is propylene glycol. In some embodiments, the formulation of the present disclosure comprises about 1% w/v to about 15% w/v propylene glycol. In some embodiments, the formulation of the present disclosure comprises about 2% w/v to about 10% w/v propylene glycol. In some embodiments, the formulation of the present disclosure comprises about 3% w/v to about 8% w/v propylene glycol.

In some embodiments, the cosolvent is glycerine. In some embodiments, the formulation of the present disclosure comprises about 2% w/v to about 30% w/v glycerine. In some embodiments, the formulation of the present disclosure comprises about 5% w/v to about 25% w/v glycerine. In some embodiment, the formulation of the present disclosure comprises about 7% w/v to about 20% w/v glycerine.

In some embodiments, the formulation of the disclosure comprises glycerine and propylene glycol as cosolvent. In some embodiments, the formulation comprises glycerine and propylene glycol, wherein the glycerine and propylene glycol is about 1% w/v to about 50% w/v of the formulation. In some embodiments, the formulation comprises glycerine and propylene glycol, wherein the glycerine and propylene glycol is about 2% w/v to about 40% w/v of the formulation. In some embodiments, the formulation comprises glycerine and propylene glycol, wherein the glycerine and propylene glycol is about 3% w/v to about 30% w/v of the formulation. In some embodiments, the formulation comprises glycerine and propylene glycol, wherein the glycerine and propylene glycol is about 3% w/v to about 25% w/v of the formulation.

In some embodiments, the formulation of the disclosure comprises glycerine and propylene glycol, wherein (i) the glycerine is about 2% w/v to about 30% w/v, about 5% w/v to about 25% w/v, or about 7% to about 20% of the formulation, and (ii) the propylene glycol is about 1% w/v to about 15% w/v, about 2% w/v to about 10% w/v, or about 3% w/v to about 8% w/v of the formulation.

In some embodiments, the formulation of the disclosure comprises glycerine and propylene glycol as cosolvent.

In some embodiments, the formulations of the present disclosure comprise at least one pharmaceutically acceptable excipient. In some embodiments, the pharmaceutically acceptable excipient comprises, but not limited to a preservative, an antioxidant, a chelating agent, a sweetener, a pH adjuster, a flavoring agent, a coloring agent, a thickening agent, or combinations thereof.

In a further embodiment, the formulations of the present disclosure comprise at least one antioxidant. In another embodiment, the formulation of the present disclosure comprises an antioxidant such as, but not limited to butylatedhydroxy anisole (BHA), butylatedhydroxy toluene (BHT), sodium metabisulfite, ascorbic acid, propyl gallate, or combination thereof. In some embodiments, the antioxidant is propyl gallate. In one embodiment, the formulation of the present disclosure comprises about 0.001% w/v to about 15% w/v antioxidant. In another embodiment, the formulation of the present disclosure comprises about 0.005% w/v to about 10% w/v antioxidant. In a further embodiment, the formulation of the present disclosure comprises about 0.005% to about 5% w/v antioxidant. In another embodiment, the formulation of the present disclosure comprises about 0.005% w/v to about 1% w/v antioxidant. In one embodiment, the formulation of the present disclosure comprises about 0.005% w/v to about 0.1% w/v antioxidant.

In one embodiment, the formulations of the present disclosure comprise at least one chelating agent. In a further embodiment, the formulation of the present disclosure comprises a chelating agent such as, but not limited to disodium edetate, dipotassium edetate, edetic acid, sodium edetate, trisodium edetate, or a combination thereof. In some embodiments, the chelating agent is disodium edetate. In another embodiment, the formulations of the present disclosure comprise about 0.001% w/v to about 10% w/v chelating agent. In still another embodiment, the formulations of the present disclosure comprise about 0.005% w/v to about 5% w/v chelating agent. In a further embodiment, the formulations of the present disclosure comprise about 0.01% w/v to about 2% w/v chelating agent.

In a further embodiment, the formulations of the present disclosure comprise at least one preservative. In one embodiment, the formulation of the present disclosure comprises preservatives such as, but not limited to sodium benzoate, methyl paraben, propylparaben, or a combination thereof. In another embodiment, the formulations of the present disclosure comprise about 0.01% w/v to about 15% w/v preservative. In yet another embodiment, the formulations of the present disclosure comprise about 0.02% w/v to about 10% w/v preservative. In a further embodiment, the formulations of the present disclosure comprise about 0.05% w/v to about 5% w/v preservative.

In one embodiment, the formulations of the present disclosure comprise at least one sweetener. In a further embodiment, the formulation of the present disclosure comprises sweetener such as but not limited to sorbitol, sucralose, acesulfame potassium, sodium saccharin or combinations thereof. In another embodiment, the formulations of the present disclosure comprise about 0.05% w/v to about 50% w/v sweetener. In still another embodiment, the formulations of the present disclosure comprise about 0.05% w/v to about 40% w/v sweetener. In a further embodiment, the formulations of the present disclosure comprise about 0.1% w/v to about 35% w/v sweetener.

In some embodiments, the sweetener is sorbitol. In some embodiments, the formulation of the present disclosure comprises about 1% w/v to about 50% w/v sorbitol. In some embodiments, the formulation of the present disclosure comprises about 2% w/v to about 40% w/v sorbitol. In some embodiments, the formulation of the present disclosure comprises about 5% w/v to about 35% w/v sorbitol.

In some embodiments, the sweetener is sucralose. In some embodiments, the formulation of the present disclosure comprises about 0.05% w/v to about 10% w/v sucralose. In some embodiments, the formulation of the present disclosure comprises about 0.05% w/v to about 5% w/v sucralose. In some embodiment, the formulation of the present disclosure comprises about 0.1% w/v to about 2% w/v sucralose.

In some embodiments, the formulation of the disclosure comprises sorbitol and sucralose as sweeteners. In some embodiments, the formulation comprises sorbitol and sucralose, wherein the sorbitol and sucralose is about 0.05% w/v to about 50% w/v of the formulation. In some embodiments, the formulation comprises sorbitol and sucralose, wherein the sorbitol and sucralose is about 0.05% w/v to about 40% w/v of the formulation. In some embodiments, the formulation comprises sorbitol and sucralose, wherein the sorbitol and sucralose is about 0.1% w/v to about 35% w/v of the formulation.

In a further embodiment, the formulations of the present disclosure comprise at least one pH adjuster. In some embodiments, the pH adjuster is an acid. In some embodiments, the acid has at least one pKa value of less than 5. In some embodiments, the acid has at least one pKa value of less than 4. In some embodiments, the acid has at least one pKa value of about 1.0 to about 3.7. In one embodiment, the pH adjuster is a phosphoric acid. In a further embodiment, the phosphoric acid is ortho-phosphoric acid. In another embodiment, the pH adjuster is present in the formulations of the present disclosure at about 0.001% w/v to about 5% w/v. In yet another embodiment, the pH adjuster is present in the formulations of the present disclosure at about 0.005% w/v to about 4% w/v. In a further embodiment, the pH adjuster is present in the formulations of the present disclosure at about 0.01% w/v to about 3% w/v. In a further embodiment, the pH adjuster is present in the formulations of the present disclosure at about 0.01% w/v to about 0.5% w/v.

In another embodiment of the present disclosure, pH of the oral liquid formulation of Trazodone is in the range of about 2.5 to about 5.5. In a further embodiment of the present disclosure, pH of the oral liquid formulation of Trazodone is in the range of about 3.0 to about 5.5. In one embodiment of the present disclosure, pH of the oral liquid formulation of Trazodone is in the range of about 3.2 to about 5.2. In another embodiment of the present disclosure, pH of the oral liquid formulation of Trazodone is in the range of about 3.8 to about 4.8. In one embodiment, the formulations of the present disclosure are buffer-free. The present disclosure demonstrates that oral liquid formulations within the claimed pH ranges provide for increased stability of trazodone and/or less precipitation, without the use of buffers. That buffer-free formulations can demonstrate increased stability and shelf-life was unexpected.

In a further embodiment, the formulations of the present disclosure may optionally comprise a flavouring agent. In another embodiment, the formulations of the present disclosure may optionally comprise a colouring agent. In one embodiment, the formulations of the present disclosure may optionally comprise a thickening agent. In one embodiment, the formulation of the present invention is in the form of solution for oral administration. In one embodiment, the formulation of the present invention is in the form of syrup for oral administration.

In one embodiment the stable oral liquid formulation of the present disclosure comprising trazodone or pharmaceutically acceptable salts thereof, one or more sweeteners, one or more preservatives and water are buffer-free. In another embodiment the stable oral liquid formulation of the present disclosure comprising trazodone or pharmaceutically acceptable salts thereof, one or more sweeteners, one or more preservatives and water do not comprise polyglycol. In a further embodiment the stable oral liquid formulation of the present disclosure comprising trazodone or pharmaceutically acceptable salts thereof, one or more sweeteners, one or more preservatives and water do not comprise polyethylene glycol. In one embodiment the stable oral liquid formulation of the present disclosure comprising trazodone or pharmaceutically acceptable salts thereof, a non-polymeric cosolvent, a pH adjuster and water are buffer-free. In another embodiment the stable oral liquid formulation of the present disclosure comprising trazodone or pharmaceutically acceptable salts thereof, a non-polymeric cosolvent, a pH adjuster and water do not comprise polyglycol. In a further embodiment the stable oral liquid formulation of the present disclosure comprising trazodone or pharmaceutically acceptable salts thereof, a non-polymeric cosolvent, a pH adjuster and water do not comprise polyethylene glycol. In one embodiment the stable oral liquid formulation of the present disclosure comprises trazodone or pharmaceutically acceptable salts thereof, one or more sweeteners, one or more preservatives, one or more cosolvents, one or more antioxidants, one or more chelating agents, one or more pH adjusters, and water. In one embodiment the stable oral liquid formulation of the present disclosure comprises about 0.1% w/v to about 10% w/v of trazodone hydrochloride, about 0.05% w/v to about 50% w/v of one or more sweeteners, about 0.01% w/v to about 15% w/v of one or more preservatives, at about 1% w/v to about 50% w/v of one or more cosolvents, about 0.001% w/v to about 15% w/v of one or more antioxidants, about 0.001% w/v to about 10% w/v of one or more chelating agents, about 0.001% w/v to about 5% w/v of one or more pH adjusters, and water. In another embodiment the stable oral liquid formulation of the present disclosure comprises about 0.1% w/v to about 10% w/v of trazodone hydrochloride, about 0.05% w/v to about 50% w/v of one or more sweeteners, about 0.01% w/v to about 15% w/v of one or more preservatives, at about 1% w/v to about 50% w/v of one or more cosolvents, about 0.001% w/v to about 15% w/v of one or more antioxidants, about 0.001% w/v to about 10% w/v of one or more chelating agents, about 0.001% w/v to about 5% w/v of one or more pH adjusters, and water in quantity sufficient to make up the volume to 100%. In a further embodiment the stable oral liquid formulation of the present disclosure comprises trazodone hydrochloride, sorbitol, sucralose, sodium benzoate, glycerine, propylene glycol, propyl gallate. disodium edetate, phosphoric acid, and water. In one embodiment the stable oral liquid formulation of the present disclosure comprises about 0.1% w/v to about 10% w/v of trazodone hydrochloride, about 0.05% w/v to about 50% w/v of sorbitol and sucralose, about 0.01% w/v to about 15% w/v of sodium benzoate, about 1% w/v to about 50% w/v of glycerine and propylene glycol, about 0.001% w/v to about 15% w/v of propyl gallate, about 0.001% w/v to about 10% w/v of disodium edetate, about 0.001% w/v to about 5% w/v of phosphoric acid, and water. In yet another embodiment the stable oral liquid formulation of the present disclosure comprises about 0.1% w/v to about 10% w/v of trazodone hydrochloride, about 0.05% w/v to about 50% w/v of sorbitol and sucralose, about 0.01% w/v to about 15% w/v of sodium benzoate, about 1% w/v to about 50% w/v of glycerine and propylene glycol, about 0.001% w/v to about 15% w/v of propyl gallate, about 0.001% w/v to about 10% w/v of disodium edetate, about 0.001% w/v to about 5% w/v of phosphoric acid, and water in quantity sufficient to make up the volume to 100%. In one embodiment the stable oral liquid formulation of the present disclosure comprises about 1% w/v of trazodone hydrochloride, about 25% w/v sorbitol, about 0.3% w/v sucralose, about 0.1% w/v sodium benzoate, about 10% w/v glycerine, about 5% w/v propylene glycol, about 0.01% w/v propyl gallate, about 0.0372% w/v disodium edetate, about 0.03% w/v of orthophosphoric acid, and water. In another embodiment the stable oral liquid formulation of the present disclosure comprises about 1% w/v of trazodone hydrochloride, about 25% w/v sorbitol, about 0.3% w/v sucralose, about 0.1% w/v sodium benzoate, about 10% w/v glycerine, about 5% w/v propylene glycol, about 0.01% w/v propyl gallate, about 0.0372% w/v disodium edetate, about 0.03% w/v of orthophosphoric acid, and water in quantity sufficient to make up the volume to 100%. In another embodiment the stable oral liquid formulation of the present disclosure comprises about 1% w/v of trazodone hydrochloride, about 25% w/v sorbitol, about 0.3% w/v sucralose, about 0.1% w/v sodium benzoate, about 15% w/v glycerine, about 5% w/v propylene glycol, about 0.01% w/v propyl gallate, about 0.0372% w/v disodium edetate, about 0.03% w/v of orthophosphoric acid, and water in quantity sufficient to make up the volume to 100%. In another embodiment the stable oral liquid formulation of the present disclosure comprises about 1% w/v of trazodone hydrochloride, about 25% w/v sorbitol, about 0.3% w/v sucralose, about 0.1% w/v sodium benzoate, about 15% w/v glycerine, about 5% w/v propylene glycol, about 0.01% w/v propyl gallate, about 0.0372% w/v disodium edetate, about 0.03% w/v of orthophosphoric acid, and water.

In one embodiment, the stable oral liquid formulation of the present disclosure comprises about 0.5% to about 2% trazodone hydrochloride, about 5% to about 15% glycerine, about 3% to about 15% propylene glycol, a phosphoric acid, and water. In another embodiment, the stable oral liquid formulation of the present disclosure comprises about 1% trazodone hydrochloride, about 10% glycerine, about 7% propylene glycol, a phosphoric acid, and water.

The present disclosure provides for trazodone oral liquid formulations with increased shelf-life. In one embodiment, the formulations of the present disclosure have a shelf-life of at least 12 months. In one embodiment, the formulations of the present disclosure have a shelf-life of at least 18 months. In another embodiment, the formulations of the present disclosure have a shelf-life of at least 2 years or 24 months. In a further embodiment, the formulations of the present disclosure have a shelf-life of at least 30 months. In yet another embodiment, the formulations of the present disclosure have a shelf-life of at least 3 years or 36 months. The term “shelf life” indicates the period over which the formulations of the present disclosure remain stable at a given storage condition.

In one embodiment, the formulation of the present disclosure provides a Cₘₐₓ of about 500 ng/mL to about 5000 ng/mL when administered to a subject under fed condition. In another embodiment, the formulation provides a Cₘₐₓ of about 500 ng/ml to about 4000 ng/ml when administered to a subject under fed condition. In another embodiment, the formulation provides a Cₘₐₓ of about 500 ng/mL to about 3000 ng/ml when administered to a subject under fed condition. Cₘₐₓ is the maximum, or peak, serum concentration of the active ingredient in the body after the active ingredient has been administered. In another embodiment, the formulation provides a Cₘₐₓ of about 500 ng/ml, about 1000 ng/ml, about 1500 ng/mL, about 2000 ng/ml, about 2500 ng/ml, about 3000 ng/mL, about 3500 ng/ml, about 4000 ng/ml, about 4500 ng/mL, or about 5000 ng/ml when administered to a subject under fed condition. The expression “fed condition” or “fed state” as used herein refers to administration of the active ingredient shortly after a meal. In some embodiments, the formulation of the present disclosure provides a Tₘₐₓ, which is the time it takes for an active ingredient to reach the maximum concentration (Cₘₐₓ) after administration, of about 0.1 hours to about 8 hours when administered to a subject under fed or fasting conditions.

In some embodiments, the formulation of the present disclosure provides AUC₀₋ₜ of about 8000 ng*hr/mL to about 54000 ng*hr/mL when administered to a subject under fed condition. In one embodiment, t as used herein refers to a time period of 24 hours. In some embodiments, the formulation provides AUC_{0-∞} of about 9000 ng*hr/mL to about 100000 ng*hr/mL when administered to a subject under fed condition. In some embodiments, the formulation provides AUC of about 9000 ng*hr/mL to about 75000 ng*hr/mL when administered to a subject under fed condition. Area under the curve (AUC) is the definite integral of the concentration of an active ingredient in blood plasma as a function of time.

In certain embodiments, the oral liquid formulation of trazodone is bioequivalent to a reference solid oral tablet.

In one embodiment, the oral liquid formulations of the present disclosure are stable at 25° C.±2° C./60%+5% RH for at least 18 months. In another embodiment, the oral liquid formulations of the present disclosure are stable at 25° C.±2° C./60%+5% RH for at least 2 years or 24 months. In a further embodiment, the formulations of the present disclosure are stable at 25° C.±2° C./60%+5% RH for 2 years or 24 months. In one embodiment, the formulations of the present disclosure are stable at 25° C.±2° C./60%+5% RH for 30 months. In yet another embodiment, the formulations of the present disclosure are stable at 25° C.±2° C./60%+5% RH for 36 months. In a further embodiment, the formulations of the present disclosure are stable at 40° C.±2° C./75%+5% RH for 6 months.

The term “stable” as used herein refers to trazodone oral liquid formulations that are free of precipitation and do not have more than about 5% w/w of total degradation products at the end of a given storage period. In some embodiments, the stable oral liquid formulations of trazodone have about 5% w/w, about 4% w/w, about 3% w/w, about 2.5% w/w, about 2% w/w, about 1.5% w/w, or about 1% w/w total degradation products. In some embodiments, the stable oral liquid formulations of trazodone have about 5% w/w total degradation products. In some embodiments, the stable oral liquid formulations of trazodone have about 4% w/w total degradation products. In some embodiments, the stable oral liquid formulations of trazodone have about 3% w/w total degradation products. In some embodiments, the stable oral liquid formulations of trazodone have about 2.5% w/w total degradation products. In some embodiments, the stable oral liquid formulations of trazodone have about 2% w/w total degradation products. In some embodiments, the stable oral liquid formulations of trazodone have about 1.5% w/w total degradation products. In some embodiments, the stable oral liquid formulations of trazodone have about 1% w/w total degradation products. In some embodiments, the given storage period is about 6 months, about 9 months, about 12 months, about 18 months, about 24 months, about 30 months or about 36 months at temperature and humidity condition of 25° C.±2° C. and 60%+5% RH. In some embodiments, the given storage period is about 6 months at temperature and humidity condition of 40° C.±2° C. and 75%+5% RH.

Stability is assessed by HPLC or any other known testing method. The term “total degradation products” as used herein refers to the total impurities in the formulations of the present invention. In some embodiments, the stable oral liquid formulations of trazodone are physically and chemically stable. In some embodiments, stable oral liquid formulations of the present invention have a shelf life at ambient temperature of equal to or greater than 18 months, 24 months, 30 months, or 36 months. In some embodiments, stable oral liquid formulations of the present invention have a shelf life at about 25° C. of equal to or greater than 18 months, 24 months, 30 months, or 36 months. In some embodiments, the formulations do not have more than about 5% w/w of total degradation products even after being stored in liquid form for an extended period of time.

In some embodiments, N-oxide impurity is not more than 0.5% in the formulations of the present disclosure stored at 25° C.±2° C./60%+5% RH for at least 18 months. In one embodiment, N-oxide impurity is not more than 0.5% in the formulations of the present disclosure stored at 25° C.±2° C./60%+5% RH for at least 2 years or 24 months. In one embodiment, N-oxide impurity is not more than 0.5% in the formulations of the present disclosure stored at 25° C.±2° C./60%+5% RH for at least 30 months. In one embodiment, N-oxide impurity is not more than 0.5% in the formulations of the present disclosure stored at 25° C.±2° C./60%+5% RH for at least 36 months. In one embodiment, N-oxide impurity is not more than 0.2% in the formulations of the present disclosure stored at 25° C.±2° C./60%+5% RH for 18 months. In another embodiment, N-oxide impurity is not more than 0.2% in the formulations of the present disclosure stored at 25° C.±2° C./60%+5% RH for 2 years or 24 months. In a further embodiment, N-oxide impurity is not more than 0.2% in the formulations of the present disclosure stored at 25° C.±2° C./60%+5% RH for 30 months. In another embodiment, N-oxide impurity is not more than 0.2% in the formulations of the present disclosure stored at 25° C./60% RH for 36 months. In one embodiment, N-oxide impurity is not more than 0.5% in the formulations of the present disclosure stored at 40° C.±2° C./75%+5% RH for 6 months. In another embodiment, N-oxide impurity is not more than 0.2% in the formulations of the present disclosure stored at 40° C.±2° C./75%+5% RH for 6 months. In another embodiment, N-oxide impurity is not more than 0.5% in the formulations of the present disclosure stored at 40° C.±2° C./75%+5% RH for 3 months. In a further embodiment, N-oxide impurity is not more than 0.2% in the formulations of the present disclosure stored at 40° C.±2° C./75%+5% RH for 3 months.

In a further embodiment, the oral liquid formulations of trazodone of the present disclosure are freeze-thaw stable. The term “freeze thaw stable” refers to a formulation that can be frozen for a period of time, e.g., greater than 24 hours, 48 hours, 72 hours, 96 hours or 1 week, and then thawed without a significant increase in precipitation, and/or formation or impurities. In another embodiment, the oral liquid formulations of trazodone of the present disclosure do not form crystals or precipitate when subjected to freeze-thaw study. In a further embodiment, formulations of the present disclosure comprising orthophosphoric acid as pH adjuster are surprisingly freeze-thaw stable.

In one embodiment, the liquid compositions of the present disclosure are homogeneous. Mixing methods may be employed in the process of preparation of the formulations of the present disclosure. Mixing methods encompass any type of mixing that results in a homogeneous oral liquid formulation.

In one embodiment, the present disclosure provides a method of preparing stable oral solution of trazodone comprising (i) adding sweetener to purified water and stirring until clear solution is obtained; (ii) adding preservative to purified water under stirring in a separate vessel; (iii) adding another sweetener to the solution of step (ii); (iv) adding the chelating agent and warming the solution of step (iii) to 60-70° C. to dissolve chelating agent; (v) allowing the solution of step (iv) to cool to NMT 35° C.; (vi) adding the solution of step (v) to solution of step (i); (vii) adding cosolvent to jacketed vessel fitted with overhead stirrer; (viii) adding purified water to step (vii) under mixing followed by heating to 65° C.±10° C. (ix) adding antioxidant to step (viii) and then allowing the solution to come to NMT 35° C.; (x) adding solution of step (ix) under stirring to the solution of step (i) to which solution of step (v) has been added; (xi) mixing pH adjuster with purified water to obtain 50% w/v pH adjuster solution. (xii) adding solution of 50% w/v pH adjuster solution of step (xi) to bulk solution of step (i) to which solutions of step (v) and step (ix) have been added and record the pH of bulk; (xiii) taking another cosolvent in a separate tank and adding water to it under stirring; (xiv) adding trazodone hydrochloride in the solution of step (xiii) and mixing till clear solution is obtained; (xv) adding solution of step (xiv) under mixing to solution of step (i) to which solutions of step (v), step (ix) and step (xi) have been added; (xvi) checking the pH of the solution (range-3.2 to 5.2); (xvii) mixing and filtering the solution of step (xvi); (xviii) adjusting the volume of the filtered solution of step (xvii) using purified water in quantity sufficient to make up the volume to 100%.

In another embodiment, the present disclosure provides a method of preparing stable oral solution of trazodone comprising (i) adding sweetener to purified water and stirring until clear solution is obtained; (ii) adding preservative to purified water under stirring in a separate vessel; (iii) adding another sweetener to the solution of step (ii); (iv) adding the chelating agent and warming the solution of step (iii) to 60-70° C. to dissolve chelating agent; (v) allowing the solution of step (iv) to cool to NMT 35° C.; (vi) adding the solution of step (v) to solution of step (i); (vii) adding cosolvent to jacketed vessel fitted with overhead stirrer; (viii) adding purified water to step (vii) under mixing followed by heating to 65° C.±10° C. (ix) adding antioxidant to step (viii) and then allowing the solution to come to NMT 35° C.; (x) adding solution of step (ix) under stirring to the solution of step (i) to which solution of step (v) has been added; (xi) mixing pH adjuster with purified water to obtain 50% w/v pH adjuster solution. (xii) adding solution of 50% w/v pH adjuster solution of step (xi) to bulk solution of step (i) to which solutions of step (v) and step (ix) have been added and record the pH of bulk; (xiii) taking another cosolvent in a separate tank and adding water to it under stirring; (xiv) adding trazodone hydrochloride in the solution of step (xiii) and mixing till clear solution is obtained; (xv) adding solution of step (xiv) under mixing to solution of step (i) to which solutions of step (v), step (ix) and step (xi) have been added; (xvi) checking the pH of the solution (range-3.8 to 4.8); (xvii) mixing and filtering the solution of step (xvi); (xviii) adjusting the volume of the filtered solution of step (xvii) using purified water in quantity sufficient to make up the volume to 100%.

In a further embodiment, the present disclosure provides a method of preparing stable oral solution of trazodone comprising (i) adding sorbitol to purified water and stirring until clear solution is obtained; (ii) adding sodium benzoate to purified water under stirring in a separate vessel; (iii) adding sucralose to the solution of step (ii); (iv) adding disodium edetate and warming the solution of step (iii) to 60-70° C. to dissolve disodium edetate; (v) allowing the solution of step (iv) to cool to NMT 35° C.; (vi) adding the solution of step (v) to solution of step (i); (vii) adding propylene glycol to jacketed vessel fitted with overhead stirrer; (viii) adding purified water to step (vii) under mixing followed by heating to 65° C.±10° C. (ix) adding propyl gallate to step (viii) and then allow the solution to come tp NMT 35° C.; (x) adding solution of step (ix) under stirring to the solution of step (i) to which solution of step (v) has been added; (xi) mixing orthophosphoric acid with purified water to obtain 50% w/v orthophosphoric solution. (xii) adding solution of 50% w/v orthophosphoric acid solution of step (xi) to bulk solution of step (i) to which solutions of step (v) and step (ix) have been added and record the pH of bulk; (xiii) taking glycerine in a separate tank and adding water to it under stirring; (xiv) adding trazodone hydrochloride in the solution of step (xiii) and mixing till clear solution is obtained; (xv) adding solution of step (xiv) under mixing to solution of step (i) to which solutions of step (v), step (ix) and step (xi) have been added; (xvi) checking the pH of the solution (range-3.2 to 5.2); (xvii) mixing and filtering the solution of step (xvi); (xviii) adjusting the volume of the filtered solution of step (xvii) using purified water in quantity sufficient to make up the volume to 100%.

In another embodiment, the present disclosure provides a method of preparing stable oral solution of trazodone comprising (i) adding sorbitol to purified water and stirring until clear solution is obtained; (ii) adding sodium benzoate to purified water under stirring in a separate vessel; (iii) adding sucralose to the solution of step (ii); (iv) adding disodium edetate and warming the solution of step (iii) to 60-70° C. to dissolve disodium edetate; (v) allowing the solution of step (iv) to cool to NMT 35° C.; (vi) adding the solution of step (v) to solution of step (i); (vii) adding propylene glycol to jacketed vessel fitted with overhead stirrer; (viii) adding purified water to step (vii) under mixing followed by heating to 65° C.±10° C. (ix) adding propyl gallate to step (viii) and then allow the solution to come tp NMT 35° C.; (x) adding solution of step (ix) under stirring to the solution of step (i) to which solution of step (v) has been added; (xi) mixing orthophosphoric acid with purified water to obtain 50% w/v orthophosphoric solution. (xii) adding solution of 50% w/v orthophosphoric acid solution of step (xi) to bulk solution of step (i) to which solutions of step (v) and step (ix) have been added and record the pH of bulk; (xiii) taking glycerine in a separate tank and adding water to it under stirring; (xiv) adding trazodone hydrochloride in the solution of step (xiii) and mixing till clear solution is obtained; (xv) adding solution of step (xiv) under mixing to solution of step (i) to which solutions of step (v), step (ix) and step (xi) have been added; (xvi) checking the pH of the solution (range-3.8 to 4.8); (xvii) mixing and filtering the solution of step (xvi); (xviii) adjusting the volume of the filtered solution of step (xvii) using purified water in quantity sufficient to make up the volume to 100%.

In a further embodiment, the formulations of the present disclosure are for oral administration. In another embodiment, the formulations of the present disclosure are provided in the form of a kit comprising the formulation and an administration device. In one embodiment, the administration device provided in the kit of the present disclosure includes, but is not limited to, syringe, dosing cup or the like. In a further embodiment, the present disclosure relates to a pharmaceutical kit comprising stable oral liquid formulation of trazodone and at least one administration device. In some embodiments, optionally a set of instructions may be included in the kit.

In yet another embodiment, the present disclosure provides for method of administration of stable oral liquid formulation of trazodone comprising withdrawing the prescribed volume of the stable oral liquid formulation of trazodone using administration device and administering orally to the patient in need thereof the withdrawn volume of the formulation.

Some embodiments relate to method of treatment of a disease or condition that can be treated or prevented with trazodone or pharmaceutically acceptable salts thereof comprising administering to the subject in need thereof stable oral liquid formulations of trazodone of the present disclosure. Some embodiments relate to method of treatment of a disease or condition that can be treated or prevented with serotonin receptor antagonists and reuptake inhibitors comprising administering to the subject in need thereof stable oral liquid formulations of trazodone of the present disclosure. One embodiment relates to method of treatment of depression comprising administering to the subject in need thereof stable oral liquid formulations of trazodone of the present disclosure. The present disclosure relates to method of treatment of depression comprising administering to the subject in need thereof stable oral liquid pharmaceutical formulation comprising trazodone or pharmaceutically acceptable salts thereof, one or more sweeteners, one or more preservatives and water. Another embodiment relates to method of treatment of major depressive disorder comprising administering to the subject in need thereof stable oral liquid formulations of trazodone of the present disclosure. The present disclosure further relates to method of treatment of major depressive disorder comprising administering to the subject in need thereof stable oral liquid pharmaceutical formulation comprising trazodone or pharmaceutically acceptable salts thereof, one or more sweeteners, one or more preservatives and water. Some embodiments relate to method of treatment of sleep disorders such as but not limited to insomnia, depression-induced insomnia and the like comprising administering to the subject in need thereof stable oral liquid formulations of trazodone of the present disclosure.

Some embodiments relate to method of treatment of a disease or condition responsive to 5-hydroxy tryptamine (5-HT) receptor antagonist in a subject in need thereof, the method comprising administering the stable oral liquid formulations of trazodone of the present disclosure. In one embodiment the disease or condition can be responsive to 5-HT 2A or 5-HT 2C receptor antagonist. In some embodiments, a disease or condition responsive to 5-hydroxy tryptamine (5-HT) receptor antagonist comprises, but not limited to, depression, anxiety, Alzheimer disease, substance abuse, bulimia, fibromyalgia, post-traumatic stress disorder (PTSD), sleep disorders, insomnia, benzodiazepine and/or alcohol dependence or abuse, dementia, schizophrenia, chronic pain, diabetic neuropathy, attention deficit hyperactivity disorder, autism spectrum disorder, or obsessive-compulsive disorder.

In some embodiments, the method of treatment comprises administering the liquid pharmaceutical formulation of the present disclosure at a dose of about 25 mg/day to about 700 mg/day trazodone. In another embodiment, the formulation is administered at a dose of about 50 mg/day to about 650 mg/day trazodone. In another embodiment, the formulation is administered at a dose of about 75 mg/day to about 650 mg/day trazodone. In another embodiment, the formulation is administered at a dose of about 100 mg/day to about 600 mg/day trazodone. In another embodiment, the formulation is administered at a dose of about 5 mg/day to about 650 mg/day trazodone. In another embodiment, the formulation is administered at a dose of about 10 mg/day to about 600 mg/day trazodone.

In some embodiments, the method of treatment comprises administering the liquid pharmaceutical formulation of the present disclosure once or more than once daily. In some embodiments, the formulation is administered in fed state. In some embodiments, the formulation is administered within 2 hours of bedtime. In some embodiments, the formulation is administered through the day in divided doses. In some embodiments, about 10% to about 75% of the total daily liquid pharmaceutical formulation is administered in the morning, and about 25% to about 90% of the total daily liquid pharmaceutical formulation is administered to the subject in the evening. In some embodiments, about 10% to about 50% of the total daily liquid pharmaceutical formulation is administered in the morning, and about 50% to about 90% of the total daily liquid pharmaceutical formulation is administered to the subject in the evening. In some embodiments, the formulation is administered for a period of time, wherein the daily amount of trazodone administered to the subject is adjusted during the period of time. In some embodiments, the daily amount of trazodone is increased every 2-5 days as needed during the period of time. In some embodiments, the daily amount of trazodone is increased every 3-4 days as needed during the period of time. In some embodiments, the daily amount of trazodone is decreased every 2-5 days as needed during the period of time. In some embodiments, the daily amount of trazodone is decreased every 3-4 days as needed during the period of time. In some embodiments, the daily amount of trazodone is increased every 3-4 days as needed during the period of time, and then decreased every 3-4 days as needed during the period of time. In some embodiments, the subject is administered a liquid pharmaceutical formulation comprising not more than 150 mg/day trazodone for first period of time, and then administered a liquid pharmaceutical formulation comprising not less than 150 mg/day trazodone for a second period of time. In some embodiments, the daily amount of trazodone administered to the subject increases 5 mg/day to 100 mg/day every 1-5 days as needed during the second period of time until a maximum effective dose is achieved. In some embodiments, the daily amount of trazodone administered to the subject increases about 10 mg/day to 75 mg/day every 1-5 days as needed during the second period of time until a maximum effective dose is achieved. In some embodiments, the daily amount of trazodone administered to the subject increases about 10 mg/day to 50 mg/day every 1-5 days as needed during the second period of time until a maximum effective dose is achieved. In some embodiments, the subject is administered a liquid pharmaceutical formulation comprising trazodone for a third period of time after the second period of time. In some embodiments, the daily amount of trazodone administered to the subject decreased about 5 mg/day to 100 mg/day every 1-5 days as needed during the third period of time until a minimum effective dose is achieved. In some embodiments, the daily amount of trazodone administered to the subject decreased about 10 mg/day to 75 mg/day every 1-5 days as needed during the third period of time until a minimum effective dose is achieved. In some embodiments, the minimum effective dose is less than 150 mg/day trazodone. In some embodiments, the minimum effective dose is less than 50 mg/day trazodone.

In certain embodiments the oral liquid formulations of trazodone are used for treatment in adults or paediatric patients.

While reference to exemplary embodiments has been made herein, it will be understood by those skilled in the art that various changes may be made, and equivalents may be substituted for elements thereof without departing from the scope of the invention. In addition, many modifications may be made to adapt a particular situation or material to the teachings without departing from the essential scope thereof. Therefore, it is intended that the disclosure is not limited to the embodiments disclosed but include all embodiments falling within the scope thereof. The present disclosure is described in more detailed by way of the following examples, but scope of the present disclosure is not limited thereto.

### EXAMPLES

### Example 1—Trazodone Oral Solution Formulation (F1)

| Ingredients | % w/v |
|---|---|
| Trazodone hydrochloride | 1 |
| Glycerine | 10 |
| Propylene glycol | 5 |
| Sorbitol NF | 25 |
| Sodium Benzoate | 0.1 |
| Sucralose | 0.3 |
| Propyl gallate | 0.01 |
| Disodium Edetate | 0.0372 |
| Ortho Phosphoric Acid | 0.03 |
| Purified Water | q.s. to 100% |

Process of Preparation-Trazodone Oral Solution was prepared by (i) adding sorbitol to purified water and stirring until clear solution is obtained; (ii) adding sodium benzoate to purified water under stirring in a separate vessel; (iii) adding sucralose to the solution of step (ii); (iv) adding disodium edetate and warming the solution of step (iii) to 60-70° C. to dissolve disodium edetate; (v) allowing the solution of step (iv) to cool to NMT 35° C.; (vi) adding the solution of step (v) to solution of step (i); (vii) adding propylene glycol to jacketed vessel fitted with overhead stirrer; (viii) adding purified water to step (vii) under mixing followed by heating to 65° C.±10° C. (ix) adding propyl gallate to step (viii) and then allow the solution to come at NMT 35° C.; (x) adding solution of step (ix) under stirring to the solution of step (i) to which solution of step (v) has been added; (xi) mixing orthophosphoric acid with purified water to obtain 50% w/v orthophosphoric solution. (xii) adding solution of 50% w/v orthophosphoric acid solution of step (xi) to bulk solution of step (i) to which solutions of step (v) and step (ix) have been added and record the pH of bulk; (xiii) taking glycerine in a separate tank and adding water to it under stirring; (xiv) adding trazodone hydrochloride in the solution of step (xiv) and mixing till clear solution is obtained; (xv) adding solution of step (xiv) under mixing to solution of step (i) to which solutions of step (v), step (ix) and step (xi) have been added; (xvi) checking the pH of the solution (range-3.8 to 4.8); (xvii) mixing and filtering the solution of step (xvi); (xvii) adjusting the volume of the filtered solution of step (xvii) using purified in quantity sufficient to make up the volume to 100%.

The formulation described above was tested for chemical and physical stability at room temperature 25±2° C. and a relative humidity of 60±5% while the container comprising the formulation is stored in an upright position.

| Test | Acceptance Criteria | Initial | 3 Months | 6 Months | 9 Months | 12 Months |
|---|---|---|---|---|---|---|
| Description | Clear, colorless | Complies | Complies | Complies | Complies | Complies |
| | solution, free from | | | | | |
| | any visible foreign | | | | | |
| | and particulate | | | | | |
| | matter, free of | | | | | |
| | precipitation and | | | | | |
| | hazy mass. | | | | | |

Organic Impurities

| | | | | | | |
|---|---|---|---|---|---|---|
| N-Oxide | Not more than 0.2% | Below | Below | Below | Below | Below |
| | | LOQ | LOQ | LOQ | LOQ | LOQ |
| Total | Not more than 1.0% | Below | Below | Below | Below | Below |
| degradation | | LOQ | LOQ | LOQ | LOQ | LOQ |
| products | | | | | | |

Assay (By HPLC)

| | | | | | | |
|---|---|---|---|---|---|---|
| Assay | 90-110% | 98 | 98.4 | 98.5 | 98.5 | 101.9 |

The formulation described above was then tested with the container stored in the horizontal position. The formulation remains physically and chemically stable at least 12 months at 25±2° C. and 60±5% RH while the container containing the formulation stored in a horizontal position.

| Test | Acceptance Criteria | Initial | 3 Months | 6 Months | 9 Months | 12 Months |
|---|---|---|---|---|---|---|
| Description | Clear, colorless | Complies | Complies | Complies | Complies | Complies |
| | solution, free from | | | | | |
| | any visible foreign | | | | | |
| | and particulate | | | | | |
| | matter, free of | | | | | |
| | precipitation and hazy | | | | | |
| | mass. | | | | | |

Organic Impurities

| | | | | | | |
|---|---|---|---|---|---|---|
| N-Oxide | Not more than 0.2% | Below | Below | Below | Below | Below |
| | | LOQ | LOQ | LOQ | LOQ | LOQ |
| Total | Not more than 1.0% | Below | Below | Below | Below | Below |
| degradation | | LOQ | LOQ | LOQ | LOQ | LOQ |
| products | | | | | | |

Assay (By HPLC)

| | | | | | | |
|---|---|---|---|---|---|---|
| Assay | 90-110% | 98 | 98.3 | 97.9 | 98.7 | 101.6 |

The formulation is physically and chemically stable and freeze-thaw stable.

### Example 2—Trazodone Oral Solution Formulations (F2-F7)

Various trazodone oral solution formulations are presented below.

| | F2 | F3 | F4 | F5 | F6 | F7 |
|---|---|---|---|---|---|---|
| Ingredients | % w/v | % w/v | % w/v | % w/v | % w/v | % w/v |
| Trazodone | 1 | 1 | 1 | 1 | 1 | 1 |
| hydrochloride | | | | | | |
| Glycerine | 15 | 5 | 0 | 10 | 10 | 10 |
| Propylene glycol | 5 | 5 | 5 | 0 | 10 | 15 |
| Sorbitol NF | 25 | 25 | 25 | 25 | 25 | 25 |
| Sodium Benzoate | 0.1 | 0.1 | 0.1 | 0.1 | 0.1 | 0.1 |
| Sucralose | 0.3 | 0.3 | 0.3 | 0.3 | 0.3 | 0.3 |
| Propyl gallate | 0.01 | 0.01 | 0.01 | 0.01 | 0.01 | 0.01 |
| Disodium Edetate | 0.0372 | 0.0372 | 0.0372 | 0.0372 | 0.0372 | 0.0372 |
| Ortho Phosphoric | 0.03 | 0.03 | 0.03 | 0.03 | 0.03 | 0.03 |
| Acid | | | | | | |
| Purified Water | q.s. to | q.s. to | q.s. to | q.s. to | q.s. to | q.s. to |
| | 100% | 100% | 100% | 100% | 100% | 100% |

Process of Preparation—Trazodone Oral Solutions are prepared by process as discussed in Example 1.

### Example 3—Trazodone Oral Solution Formulation (F8)

| Ingredients | % w/v |
|---|---|
| Trazodone hydrochloride | 1 |
| Glycerine | 10 |
| Propylene glycol | 5 |
| Sorbitol NF | 20 |
| Sodium Benzoate | 0.1 |
| Sucralose | 0.3 |
| Sodium metabisulpite | 0.01 |
| Dipotassium Edetate | 0.0372 |
| Ortho Phosphoric Acid | 0.04 |
| Purified Water | q.s. to 100% |

Process of Preparation-Trazodone Oral Solution is prepared by process as discussed in Example 1.

### Example 4—Trazodone Oral Solution Formulation (F))

| Ingredients | % w/v |
|---|---|
| Trazodone hydrochloride | 1 |
| Glycerine | 12 |
| Propylene glycol | 5 |
| Sorbitol NF | 22 |
| Sodium Benzoate | 0.1 |
| Sucralose | 0.3 |
| Butylated hydroxytoluene | 0.01 |
| Disodium Edetate | 0.0372 |
| Ortho Phosphoric Acid | 0.035 |
| Purified Water | q.s. to 100% |

Process of Preparation-Trazodone Oral Solution is prepared by process as discussed in Example 1.

### Example 5—Trazodone Oral Solution Formulation (F10)

| Ingredients | % w/v |
|---|---|
| Trazodone hydrochloride | 1 |
| Glycerine | 0 |
| Propylene glycol | 5 |
| Sorbitol NF | 20 |
| Methyl paraben | 0.05 |
| Propyl paraben | 0.05 |
| Sucralose | 0.3 |
| Propyl gallate | 0.01 |
| Disodium Edetate | 0.0372 |
| Ortho Phosphoric Acid | 0.04 |

Process of Preparation-Trazodone Oral Solution is prepared by process as discussed in Example 1.

### Example 6—Trazodone Oral Solution Formulation (F11-F16)

| | F11 | F12 | F13 | F14 | F15 | F16 |
|---|---|---|---|---|---|---|
| Ingredients | % w/v | % w/v | % w/v | % w/v | % w/v | % w/v |
| Trazodone | 1 | 1 | 1 | 1 | 1 | 1 |
| hydrochloride | | | | | | |
| Glycerine | 15 | 0 | 7 | 5 | 5 | 0 |
| Propylene glycol | 0 | 15 | 7 | 15 | 10 | 15 |
| Sorbitol NF | 25 | 25 | 25 | 25 | 25 | 25 |
| Sodium Benzoate | 0.1 | 0.1 | 0.1 | 0.1 | 0.1 | 0.1 |
| Sucralose | 0.3 | 0.3 | 0.3 | 0.3 | 0.3 | 0.3 |
| Propyl gallate | 0.01 | 0.01 | 0.01 | 0.01 | 0.01 | 0.01 |
| Disodium Edetate | 0.0372 | 0.0372 | 0.0372 | 0.0372 | 0.0372 | 0.0372 |
| Ortho Phosphoric | 0.03 | 0.03 | 0.03 | 0.03 | 0.03 | 0.03 |
| Acid | | | | | | |
| Purified Water | q.s. to | q.s. to | q.s. to | q.s. to | q.s. to | q.s. to |
| | 100% | 100% | 100% | 100% | 100% | 100% |

Process of Preparation-Trazodone Oral Solutions are prepared by process as discussed in Example 1.

### Example 7—Trazodone Oral Solution Formulation (F17-F22)

| | F11 | F12 | F13 | F14 | F15 | F16 |
|---|---|---|---|---|---|---|
| Ingredients | % w/v | % w/v | % w/v | % w/v | % w/v | % w/v |
| Trazodone | 1 | 1 | 1 | 1 | 1 | 1 |
| hydrochloride | | | | | | |
| Glycerine | 15 | 5 | 0 | 10 | 10 | 10 |
| Propylene glycol | 5 | 5 | 5 | 0 | 10 | 15 |
| Sorbitol NF | 25 | 25 | 25 | 25 | 25 | 25 |
| Sodium Benzoate | 0.1 | 0.1 | 0.1 | 0.1 | 0.1 | 0.1 |
| Sucralose | 0.3 | 0.3 | 0.3 | 0.3 | 0.3 | 0.3 |
| Propyl gallate | 0.01 | 0.01 | 0.01 | 0.01 | 0.01 | 0.01 |
| Disodium Edetate | 0.0372 | 0.0372 | 0.0372 | 0.0372 | 0.0372 | 0.0372 |
| Ortho-Phosphoric acid | 0.04 | 0.035 | 0.045 | 0.05 | 0.044 | 0.048 |
| Purified Water | q.s. to | q.s. to | q.s. to | q.s. to | q.s. to | q.s. to |
| | 100% | 100% | 100% | 100% | 100% | 100% |

Process of Preparation-Trazodone Oral Solutions are prepared by process as discussed in Example 1.

### Example 8—Trazodone Oral Solution Formulation (F23)

| Ingredients | % w/v |
|---|---|
| Trazodone hydrochloride | 1 |
| Glycerine | 15 |
| Propylene glycol | 7 |
| Sorbitol NF | 25 |
| Sodium Benzoate | 0.1 |
| Sucralose | 0.3 |
| Propyl gallate | 0.01 |
| Disodium Edetate | 0.0372 |
| Ortho Phosphoric Acid | 0.03 |
| Purified Water | q.s. to 100% |

Process of Preparation-Trazodone Oral Solution was prepared by (i) adding sorbitol to purified water and stirring until clear solution is obtained; (ii) adding sodium benzoate to purified water under stirring in a separate vessel; (iii) adding sucralose to the solution of step (ii); (iv) adding disodium edetate and warming the solution of step (iii) to 60-70° C. to dissolve disodium edetate; (v) allowing the solution of step (iv) to cool to NMT 35° C.; (vi) adding the solution of step (v) to solution of step (i); (vii) adding propylene glycol to in jacketed vessel fitted with overhead stirrer; (viii) adding purified water to step (vii) under mixing followed by heating to 65° C.±10° C. (ix) adding propyl gallate to step (viii) and then allow the solution to come at NMT 35° C.; (x) adding solution of step (ix) under stirring to the solution of step (i) to which solution of step (v) has been added; (xi) mixing orthophosphoric acid with purified water to obtain 50% w/v orthophosphoric solution; (xii) adding solution of 50% w/v orthophosphoric acid solution of step (xi) to bulk solution of step (i) to which solutions of step (v) and step (ix) have been added and record the pH of bulk; (xiii) taking glycerine in a separate tank and adding water to it under stirring; (xiv) adding trazodone hydrochloride in the solution of step (xiv) and mixing till clear solution is obtained; (xv) adding solution of step (xiv) under mixing to solution of step (i) to which solutions of step (v), step (ix) and step (xi) have been added; (xvi) checking the pH of the solution (range-3.8 to 4.8); (xvii) mixing and filtering of the solution of step (xvi); (xviii) adjusting the volume of the filtered solution of step (xvii) using purified water in quantity sufficient to make up the volume to 100%.

The formulation is physically and chemically stable and freeze-thaw stable.

1. A liquid pharmaceutical formulation comprising:
(i) trazodone or a pharmaceutically acceptable salt thereof;
(ii) a non-polymeric cosolvent;
(iii) a pH adjuster; and
(iv) water;
wherein the formulation does not comprise polyethylene glycol, and wherein the pH of the formulation is about 2.5 to about 5.5.

2. The formulation of claim 1, wherein the trazodone is trazodone hydrochloride.

3. The formulation of claim 1, wherein the trazodone is about 0.1% w/v to about 10% w/v of the formulation

4. The formulation of claim 3, wherein the trazodone is about 0.5% w/v to about 5% w/v of the formulation.

5. The formulation of claim 4, wherein the trazodone is about 0.8% w/v to about 1.2% w/v of the formulation.

6. The formulation of claim 1, wherein the non-polymeric cosolvent is polyol.

7. The formulation of claim 1, wherein the non-polymeric cosolvent comprises propylene glycol, glycerine, or combinations thereof.

8. The formulation of claim 1, wherein the non-polymeric cosolvent is about 1% w/v to about 50% w/v of the formulation.

9. The formulation of claim 8, wherein the non-polymeric cosolvent is about 3% w/v to about 30% w/v of the formulation.

10. The formulation of claim 1, wherein the formulation does not comprise a polymeric cosolvent.

11. The formulation of claim 1, wherein the non-polymeric cosolvent comprises propylene glycol.

12. The formulation of claim 11, wherein the propylene glycol is about 1% w/v to about 15% w/v of the formulation.

13. The formulation of claim 1, wherein the non-polymeric cosolvent comprises glycerine.

14. The formulation of claim 13, wherein the glycerine is about 2% w/v to about 30% w/v of the formulation.

15. The formulation of claim 1, wherein the non-polymeric cosolvent comprises a combination of propylene glycol and glycerine.

16. The formulation of claim 1, wherein the pH adjuster is an acid.

17. The formulation of claim 16, wherein the acid has at least one pKa value of less than 5.

18. The formulation of claim 16, wherein the acid has at least one pKa value of about 1.0 to about 3.7.

19. The formulation of claim 1, wherein the pH adjuster is a phosphoric acid.

20. The formulation of claim 19, wherein the phosphoric acid is ortho-phosphoric acid.

21. The formulation of claim 1, wherein the pH adjuster is about 0.01% w/v to about 3% w/v of the formulation.

22. The formulation of claim 21, wherein the pH adjuster is about 0.01% w/v to about 0.5% w/v of the formulation.

23. The formulation of claim 1, wherein the pH of the formulation is about 3.8 to about 4.8.

24. The formulation of claim 1, wherein the formulation further comprises at least one pharmaceutically acceptable excipient.

25. The formulation of claim 24, wherein said pharmaceutically acceptable excipient comprises a preservative, an antioxidant, a chelating agent, a sweetener, a flavoring agent, a coloring agent, a thickening agent, or combinations thereof.

26. The formulation of claim 25, wherein the antioxidant comprises butylatedhydroxy anisole (BHA), butylatedhydroxy toluene (BHT), sodium metabisulfite, ascorbic acid, propyl gallate, or combination thereof.

27. The formulation of claim 25, wherein the antioxidant comprises propyl gallate.

28. The formulation of claim 25, wherein the chelating agent comprises disodium edetate, dipotassium edetate, edetic acid, sodium edetate, trisodium edetate, or a combination thereof.

29. The formulation of claim 25, wherein the chelating agent comprises disodium edetate.

30. The formulation of claim 25, wherein the preservative comprises sodium benzoate, methyl paraben, propylparaben, or combinations thereof.

31. The formulation of claim 25, wherein the sweetener is sorbitol, sucralose, acesulfame potassium, sodium saccharin, or combinations thereof.

32. The formulation of claim 1, wherein the formulation is stable for greater than 18 months.

33. The formulation of claim 1, wherein the formulation is freeze-thaw stable.

34. A liquid pharmaceutical formulation comprising:
(i) about 0.5% to about 2% trazodone hydrochloride;
(ii) about 5% to about 15% glycerine;
(iii) about 3% to about 15% propylene glycol;
(iv) a phosphoric acid; and
(v) water;
wherein the formulation does not comprise a polymeric cosolvent and wherein the pH of the formulation is about 3.8 to about 4.8.

35. A liquid pharmaceutical formulation comprising:
(i) about 1% trazodone hydrochloride;
(ii) about 10% glycerine;
(iii) about 5% propylene glycol;
(iv) a phosphoric acid; and
(v) water;
wherein the formulation does not comprise a polymeric cosolvent and wherein the pH of the formulation is about 3.8 to about 4.8.

36. A liquid pharmaceutical formulation comprising:
(i) about 1% trazodone hydrochloride;
(ii) about 15% glycerine;
(iii) about 7% propylene glycol;
(iv) a phosphoric acid; and
(v) water;
wherein the formulation does not comprise a polymeric cosolvent and wherein the pH of the formulation is about 3.8 to about 4.8.

37. The formulation of claim 34, wherein the formulation comprises 100 mg/ml trazodone, and wherein the formulation is stable for at least 12 months.

38. The formulation of claim 34, wherein the formulation is stable for at least 18 months.

39. The formulation of claim 34, wherein the formulation is stable for at least 24 months.

40. The formulation of claim 34, wherein the formulation comprises 100 mg/ml trazodone, and wherein the formulation provides a Cₘₐₓ of about 500 ng/ml to about 5000 ng/mL when administered to a subject under fed condition.

41. The formulation of claim 34, wherein the formulation comprises 100 mg/ml trazodone, and wherein the formulation provides a AUC₀₋ₜ of about 8000 ng*hr/mL to about 54000 ng*hr/mL when administered to a subject under fed condition.

42. The formulation of claim 34, wherein the formulation comprises 100 mg/ml trazodone, and wherein the formulation provides a AUC_{0-∞} of about 9000 ng*hr/mL to about 100000 ng*hr/mL when administered to a subject under fed condition.

43. The formulation of claim 34, wherein the formulation comprises 100 mg/ml trazodone, and wherein the formulation provides Tₘₐₓ of about 0.1 hours to about 8 hours when administered to a subject under fed or fasting conditions.

44. A method of treating a depressive disorder in a subject in need thereof, the method comprising administering a liquid pharmaceutical formulation comprising:
(i) trazodone or a pharmaceutically acceptable salt thereof;
(ii) a cosolvent;
(iii) a pH adjuster; and
(iv) water;
wherein the formulation does not comprise polyethylene glycol, and wherein the pH of the formulation is about 2.5 to about 5.5.

45. The method of claim 44, wherein the depressive disorder is a major depressive disorder.

46. The method of claim 44, wherein the liquid pharmaceutical formulation is administered at a dose of about 25 mg/day to about 700 mg/day trazodone.

47. The method of claim 46, wherein the liquid pharmaceutical formulation is administered at a dose of about 50 mg/day to about 650 mg/day trazodone.

48. The method of claim 47, wherein the liquid pharmaceutical formulation is administered at a dose of about 75 mg/day to about 650 mg/day trazodone.

49. The method of claim 48, wherein the liquid pharmaceutical formulation is administered at a dose of about 100 mg/day to about 600 mg/day trazodone.

50. A method of treating a disease or condition responsive to 5-hydroxy tryptamine (5-HT) receptor antagonist in a subject in need thereof, the method comprising administering a liquid pharmaceutical formulation comprising:
(i) trazodone or a pharmaceutically acceptable salt thereof;
(ii) a cosolvent;
(iii) a pH adjuster; and
(iv) water;
wherein the formulation does not comprise polyethylene glycol, and wherein the pH of the formulation is about 2.5 to about 5.5.

51. The method of claim 50, wherein the liquid pharmaceutical formulation is administered at a dose of about 5 mg/day to about 650 mg/day trazodone.

52. The method of claim 51, wherein the liquid pharmaceutical formulation is administered at a dose of about 10 mg/day to about 600 mg/day trazodone.

53. The method of claim 50, wherein the disease or condition responsive to 5-hydroxy tryptamine (5-HT) receptor antagonist comprises depression, anxiety, Alzheimer disease, substance abuse, bulimia, fibromyalgia, post-traumatic stress disorder (PTSD), sleep disorders, insomnia, benzodiazepine and/or alcohol dependence or abuse, dementia, schizophrenia, chronic pain, diabetic neuropathy, attention deficit hyperactivity disorder, autism spectrum disorder, or obsessive-compulsive disorder.

54. The method of claim 44, wherein the liquid pharmaceutical formulation is administered once or more than once daily.

55. The method of claim 44, wherein the liquid pharmaceutical formulation is administered in fed state.

56. The method of claim 44, wherein the liquid pharmaceutical formulation is administered in within 2 hours of bedtime.

57. The method of claim 44, wherein the liquid pharmaceutical formulation is administered through the day in divided doses.

58. The method of claim 44, wherein about 10% to about 75% of the total daily liquid pharmaceutical formulation is administered in the morning, and about 25% to about 90% of the total daily liquid pharmaceutical formulation is administered to the subject in the evening.

59. The method of claim 44, wherein about 10% to about 50% of the total daily liquid pharmaceutical formulation is administered in the morning, and about 50% to about 90% of the total daily liquid pharmaceutical formulation is administered to the subject in the evening.

60. The method of claim 44, wherein the liquid pharmaceutical formulation is administered for a period of time, wherein the daily amount of trazodone administered to the subject is adjusted during the period of time.

61. The method of claim 60, wherein the daily amount of trazodone is increased every 2-5 days as needed during the period of time.

62. The method of claim 60, wherein the daily amount of trazodone is increased every 3-4 days as needed during the period of time.

63. The method of claim 60, wherein the daily amount of trazodone is decreased every 2-5 days as needed during the period of time.

64. The method of claim 60, wherein the daily amount of trazodone is decreased every 3-4 days as needed during the period of time.

65. The method of claim 60, wherein the daily amount of trazodone is increased every 3-4 days as needed during the period of time, and then decreased every 3-4 days as needed during the period of time.

66. The method of claim 60, wherein the subject is administered a liquid pharmaceutical formulation comprising not more than 150 mg/day trazodone for first period of time, and then administered a liquid pharmaceutical formulation comprising not less than 150 mg/day trazodone for a second period of time.

67. The method of claim 66, wherein the daily amount of trazodone administered to the subject increases 5 mg/day to 100 mg/day every 1-5 days as needed during the second period of time until a maximum effective dose is achieved.

68. The method of claim 66, wherein the daily amount of trazodone administered to the subject increases about 10 mg/day to 75 mg/day every 1-5 days as needed during the second period of time until a maximum effective dose is achieved.

69. The method of claim 66, wherein the daily amount of trazodone administered to the subject increases about 10 mg/day to 50 mg/day every 1-5 days as needed during the second period of time until a maximum effective dose is achieved.

70. The method of claim 67, wherein the daily amount of trazodone administered to the subject increases about 10 mg/day to 50 mg/day every 1-5 days as needed during the second period of time until a maximum effective dose is achieved.

71. The method of claim 70, wherein the daily amount of trazodone administered to the subject decreased about 5 mg/day to 100 mg/day every 1-5 days as needed during the third period of time until a minimum effective dose is achieved.

72. The method of claim 70, wherein the daily amount of trazodone administered to the subject decreased about 10 mg/day to 75 mg/day every 1-5 days as needed during the third period of time until a minimum effective dose is achieved.

73. The method of claim 71, wherein the minimum effective dose is less than 150 mg/day trazodone.

74. The method of claim 73, wherein the minimum effective dose is less than 50 mg/day trazodone.
